(19) United States
(12) Patent Application Publication
(10) Pub. No.: US 2026/0259119 A1
(43) Pub. Date: Sep. 3, 2026

(54) SIMULATION DEVICE AND SIMULATION METHOD FOR CONSOLIDATION AND SETTLEMENT WITH VARIABLE CONFINEMENT CONSTRAINT

(71) Applicant: CHINA UNIVERSITY OF MINING AND TECHNOLOGY, Jiangsu (CN)

(72) Inventors: Shuzhao CHEN, Jiangsu (CN); Quansheng LI, Jiangsu (CN); Xinyu GENG, Jiangsu (CN); Daoyan SUN, Jiangsu (CN); Fan JIANG, Jiangsu (CN); Hai WANG, Jiangsu (CN)

(21) Appl. No.: 19/534,071

(22) Filed: Feb. 9, 2026

(30) Foreign Application Priority Data
Mar. 3, 2025 (CN) 202510239237.3

(51) Int. Cl. G01N 3/10; G01N 3/06; G01N 33/24

(52) U.S. Cl. CPC G01N 3/10; G01N 3/06; G01N 33/24; G01N 2203/0284

(57) ABSTRACT

Disclosed are a simulation device and a simulation method for consolidation and settlement with a variable confinement constraint. The simulation device includes a pressure cover, an outer frame, and an inner test area, and can separately carry out a consolidation test without lateral deformation, a pressure settlement test with a weak constraint in a single direction or without a constraint, a pressure settlement test with variable constraints in multiple directions, and a stress transfer test. The simulation device is divided into an inner layer and an outer layer, so that the rigidity of the simulation device is improved and the impact of the deformation of the simulation device on the test is further reduced.

### CROSS REFERENCE TO THE RELATED APPLICATIONS

This application is based upon and claims priority to Chinese Patent Application No. 202510239237.3, filed on March 3, 2025, the entire contents of which are incorporated herein by reference.

### TECHNICAL FIELD

The present disclosure relates to a simulation device, and in particular, to a simulation device and a simulation method for consolidation and settlement with a variable confinement constraint.

### BACKGROUND

The settlement of dumps of open-pit mines affects the safe production of mines and the ecological restoration of the dumps in the later stages. Accurate prediction of the direction and extent of settlement is of great significance for open-pit mines. All conventional compaction and consolidation test benches adopt rigid, uniform constraints, i.e., the four sides and bottom of the test bench are rigid and cannot undergo displacement. However, the settlement of dumps of open-pit mines involves more than one displacement direction due to geological conditions, bench shape, goaf location, and other factors. Consequently, conventional compaction and consolidation test benches cannot accurately simulate dump settlement, directly affecting the rational determination of the safe height of the bench and the calculation of the effective capacity of the dump.

### SUMMARY

To solve the problems in the prior art, the present disclosure provides a simulation device and a simulation method for consolidation and settlement with a variable confinement constraint, to set up a simulation scene that more closely resembles the real situation of a dump of an open-pit mine, thereby achieving a more realistic simulation result.

To achieve the above objective, the present disclosure provides the following technical solutions. A simulation device for consolidation and settlement with a variable confinement constraint is provided, including: an outer front plate, an outer rear plate, an outer left side plate, an outer right side plate, and a bottom plate, where a left edge and a right edge of the outer front plate are respectively connected to a front edge of the outer left side plate and a front edge of the outer right side plate, a left edge and a right edge of the outer rear plate are respectively connected to a rear edge of the outer left side plate and a rear edge of the outer right side plate, a bottom edge of the outer front plate, a bottom edge of the outer rear plate, a bottom edge of the outer left side plate, and a bottom edge of the outer right side plate are connected to a top surface of the bottom plate, and the outer front plate, the outer rear plate, the outer left side plate, and the outer right side plate form an outer side frame; and

the simulation device for consolidation and settlement with the variable confinement constraint further includes a pressure cover, an inner front plate, an inner rear plate, an inner left side plate, and an inner right side plate, where the inner rear plate is located on an inner side of the outer rear plate, a left edge and a right edge of the inner rear plate are respectively connected to the outer left side plate and the outer right side plate, a bottom edge of the inner rear plate is connected to the top surface of the bottom plate, and the inner rear plate and the outer rear plate are rigidly connected by a supporting column;

inner side-plate slide rails are respectively arranged at positions on an inner side surface of the inner rear plate adjacent to the left edge and the right edge of the inner rear plate and positions on an inner side surface of the outer front plate adjacent to the left edge and the right edge of the outer front plate, and through the inner side-plate slide rails, a front edge and a rear edge of the inner left side plate are respectively connected to the outer front plate and the inner rear plate, and a front edge and a rear edge of the inner right side plate are respectively connected to the outer front plate and the inner rear plate;

inner front-plate slide rails are respectively arranged at a position on an inner side surface of the inner left side plate adjacent to the outer front plate and a position on an inner side surface of the inner right side plate adjacent to the outer front plate, a telescopic cylinder is arranged inside the inner front plate, two telescopic bars are respectively arranged at a left end and a right end of the telescopic cylinder, and the two telescopic bars at the left end and the right end of the telescopic cylinder are respectively connected to the inner left side plate and the inner right side plate through the inner front-plate slide rails;

a bottom edge of the inner front plate, a bottom edge of the inner left side plate, and a bottom edge of the inner right side plate are in contact with the bottom plate, a test area is defined by the inner rear plate, the inner front plate, the inner left side plate, and the inner right side plate, and pressure sensors are arranged on the bottom plate within the test area;

a first hydraulic cylinder and a first limiting block are arranged between the inner front plate and the outer front plate, a second hydraulic cylinder and a second limiting block are arranged between the inner left side plate and the outer left side plate, a third hydraulic cylinder and a third limiting block are arranged between the inner right side plate and the outer right side plate, limiting grooves are respectively provided on the inner side surface of the outer front plate, an inner side surface of the outer left side plate, and an inner side surface of the outer right side plate, and on an outer side surface of the inner front plate, an outer side surface of the inner left side plate, and an outer side surface of the inner right side plate, and two ends of each of the first limiting block, the second limiting block, and the third limiting block are respectively engaged with two respective ones of the limiting grooves; and

the pressure cover includes a cover body, a central disc, a pressure column, and a pressure plate, a shape and a size of the cover body match a shape and a size of the outer side frame, the central disc is arranged at a center of the cover body, an upper end and a lower end of the pressure column are respectively connected to a lower end surface of the central disc and an upper end surface of the pressure plate, the pressure plate is located above the test area, and the pressure cover is detachably connected to the outer side frame.

Further, a reinforcing column is arranged at each of four inner corners of the outer side frame.

Further, four first connecting columns are respectively arranged at a middle position on the inner side surface of the outer front plate, a middle position on an inner side surface of the outer rear plate, a middle position on the inner side surface of the outer left side plate, and a middle position on the inner side surface of the outer right side plate.

Further, four second connecting columns are respectively arranged at middle positions on four edges of a lower end surface of the cover body, and the four second connecting columns on the lower end surface of the cover body are respectively connected to the four first connecting columns of the outer side frame by connecting buckles.

Further, two telescopic cylinders are provided and spaced apart in a vertical direction, and a plurality of first hydraulic cylinders and a plurality of first limiting blocks are arranged and spaced apart between the inner front plate and the outer front plate, a plurality of second hydraulic cylinders and a plurality of second limiting blocks are arranged and spaced apart between the inner left side plate and the outer left side plate, and a plurality of third hydraulic cylinders and a plurality of third limiting blocks are arranged and spaced apart between the inner right side plate and the outer right side plate.

Further, a lifting ring is arranged on an upper top surface of the central disc.

Further, the pressure sensors are arranged in an array on the bottom plate within the test area.

Further, the pressure plate and the pressure column are connected by a snap-fit, and the pressure plate is circular, square, or rectangular depending on test requirements.

A simulation method for consolidation and settlement with a variable confinement constraint is provided, including:

simulating a consolidation test without lateral deformation: applying a vertical pressure to a material by a pressure column, and recording a relationship between the vertical pressure from the pressure column, a compression amount of the material, and a duration for which the vertical pressure is applied to the material;

simulating a pressure settlement test with a weak constraint in a single direction or without a constraint: taking out a first limiting block between an inner front plate and an outer front plate, setting a pressure upper limit for a first hydraulic cylinder between the inner front plate and the outer front plate, applying a vertical pressure to a material by the pressure column, and recording a relationship between the vertical pressure from the pressure column, a compression amount of the material, a displacement of the inner front plate, pressure values of pressure sensors on a bottom plate, and a duration for which the vertical pressure is applied to the material;

simulating a pressure settlement test with variable constraints in multiple directions: taking out the first limiting block between the inner front plate and the outer front plate, a second limiting block between an inner left side plate and an outer left side plate, and a third limiting block between an inner right side plate and an outer right side plate, setting an upper limit for support forces from the first hydraulic cylinder between the inner front plate and the outer front plate, a second hydraulic cylinder between the inner left side plate and the outer left side plate, and a third hydraulic cylinder between the inner right side plate and the outer right side plate, applying a vertical pressure to a material by the pressure column, and recording the vertical pressure from the pressure column, a compression amount of the material, a pressure received by the inner front plate, a horizontal displacement of the inner front plate, a pressure received by the inner left side plate, a horizontal displacement of the inner left side plate, a pressure received by the inner right side plate, a horizontal displacement of the inner right side plate, displacements of the first hydraulic cylinder, the second hydraulic cylinder and the third hydraulic cylinder, pressure values of the pressure sensors on the bottom plate, and a duration for which the vertical pressure is applied to the material; and

simulating a stress transfer test: taking out the first limiting block between the inner front plate and the outer front plate or taking out the second limiting block between the inner left side plate and the outer left side plate and the third limiting block between the inner right side plate and the outer right side plate, setting a displacement limit for the inner front plate or for the inner left side plate and the inner right side plate in advance, applying a vertical pressure to a material by the pressure column, recording a relationship between the vertical pressure from the pressure column, a compression amount of the material, a pressure received by the inner front plate or a pressure received by the inner left side plate and the inner right side plate, pressure values of the pressure sensors on the bottom plate, and a duration for which the vertical pressure is applied to the material.

Compared with the prior art, in the present disclosure, the simulation device is divided into an inner layer and an outer layer, so that the rigidity of the simulation device is improved and the impact of the deformation of the simulation device on the test is further reduced. The inner plates are designed to be slidable, so that the relationship between pressure, compression amount, displacement, bottom pressure, time, and other factors can be simulated separately or synchronously in the case of 0 to 3 free surfaces of the dump, thereby setting up a simulation scene that more closely resembles the real situation of a dump of an open-pit mine. Before the test, the material is loaded, and pressure and displacement limits are set for the hydraulic cylinders. The test process is simple to operate, requires only an action of controlling the pressure column, with other parameters being automatically collected, and can achieve high measurement precision and a reliable simulation result. The simulation device has a skillful and simple design using steel structures, hydraulic systems, connecting members, and other components, and uses readily available materials. The simulation device is easy to manufacture, and is suitable for large-scale production.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a structural cross-sectional top view of the present disclosure.

FIG. 2 is a structural cross-sectional side view of the present disclosure.

FIG. 3 is a top view of a bottom plate of the present disclosure.

### DETAILED DESCRIPTION OF THE EMBODIMENTS

The present disclosure is further described below with reference to the accompanying drawings.

The technical solutions in the embodiments of the present disclosure will be described clearly and fully with reference to the accompanying drawings in the embodiments of the present disclosure. Apparently, the embodiments described are merely some embodiments, rather than all of the embodiments of the present disclosure. All other embodiments obtained by those of ordinary skill in the art without creative efforts based on the embodiments of the present disclosure shall fall within the protection scope of the present disclosure.

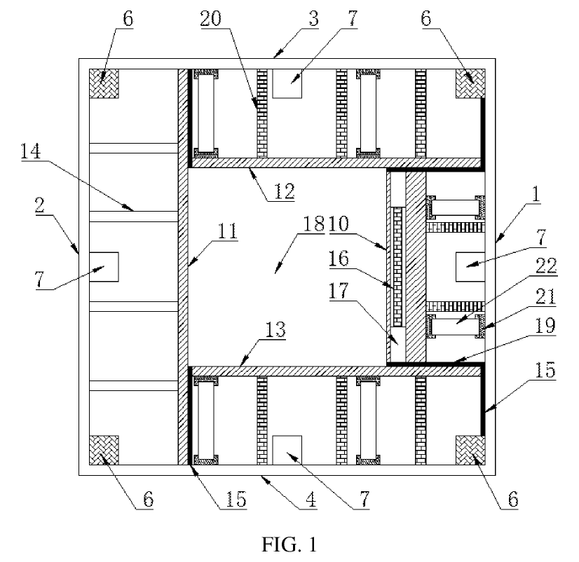

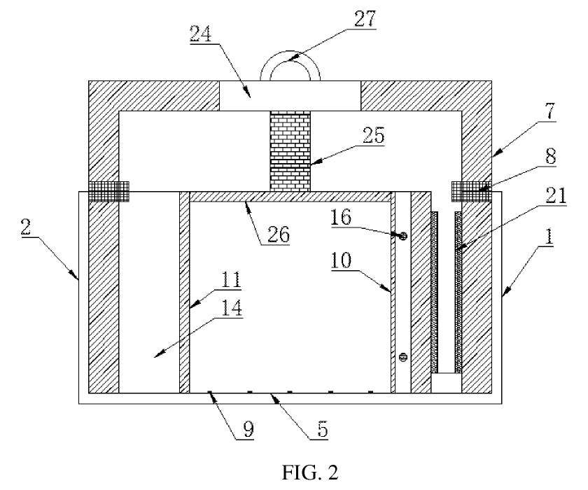

As shown in FIG. 1 to FIG. 2, the present disclosure provides a simulation device for consolidation and settlement with a variable confinement constraint. The simulation device includes a pressure cover, an outer frame, and an inner test area. The outer frame includes an outer front plate 1, an outer rear plate 2, an outer left side plate 3, an outer right side plate 4, and a bottom plate 5. A left edge and a right edge of the outer front plate 1 are respectively connected to a front edge of the outer left side plate 3 and a front edge of the outer right side plate 4, and a left edge and a right edge of the outer rear plate 2 are respectively connected to a rear edge of the outer left side plate 3 and a rear edge of the outer right side plate 4. A bottom edge of the outer front plate 1, a bottom edge of the outer rear plate 2, a bottom edge of the outer left side plate 3, and a bottom edge of the outer right side plate 4 are connected to a top surface of the bottom plate 5. The outer front plate 1, the outer rear plate 2, the outer left side plate 3, and the outer right side plate 4 form an outer side frame of a steel structure, which mainly serves as a support. A reinforcing column 6 is arranged at each of four inner corners of the outer side frame to further strengthen the support. Four first connecting columns 7 are respectively arranged at a middle position on an inner side surface of the outer front plate 1, a middle position on an inner side surface of the outer rear plate 2, a middle position on an inner side surface of the outer left side plate 3, and a middle position on an inner side surface of the outer right side plate 4.

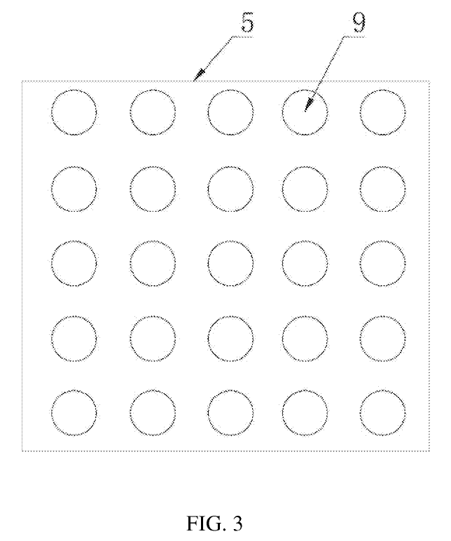

The inner test area includes an inner front plate 10, an inner rear plate 11, an inner left side plate 12, and an inner right side plate 13. The inner rear plate 11 is located on an inner side of the outer rear plate 2. A left edge and a right edge of the inner rear plate 11 are respectively connected to the outer left side plate 3 and the outer right side plate 4. A bottom edge of the inner rear plate 11 is connected to the top surface of the bottom plate 5. The inner rear plate 11 and the outer rear plate 2 are rigidly connected by a supporting column 14. Inner side-plate slide rails 15 are respectively arranged at positions on an inner side surface of the inner rear plate 11 adjacent to the left edge and the right edge of the inner rear plate 11 and positions on the inner side surface of the outer front plate 1 adjacent to the left edge and the right edge of the outer front plate 1. Through the inner side-plate slide rails 15, a front edge and a rear edge of the inner left side plate 12 are respectively connected to the outer front plate 1 and the inner rear plate 11, and a front edge and a rear edge of the inner right side plate 13 are respectively connected to the outer front plate 1 and the inner rear plate 11, so that the inner left side plate 12 and the inner right side plate 13 can be moved along the inner side-plate slide rails 15. Inner front-plate slide rails 19 are respectively arranged at a position on an inner side surface of the inner left side plate 12 adjacent to the outer front plate 1 and a position on an inner side surface of the inner right side plate 13 adjacent to the outer front plate 1. The inner front plate 10 is a composite steel plate structure. Two telescopic cylinders 16 are provided and spaced apart in a vertical direction inside the inner front plate 10. Two telescopic bars 17 are respectively arranged at a left end and a right end of each of the two telescopic cylinders 16. The two telescopic bars 17 at the left end and the right end of each of the two telescopic cylinders 16 are respectively connected to the inner left side plate 12 and the inner right side plate 13 through the inner front-plate slide rails 19, so that the inner front plate 10 can be moved along the inner front-plate slide rails 19. The telescopic bars 17 can be extended along with the displacement of the inner left side plate 12 and the inner right side plate 13, to ensure that the test area 18 is sealed. The telescopic cylinders 16 respectively record displacements of the corresponding telescopic bars 17, and help the corresponding telescopic bars 17, the inner left side plate 12, and the inner right side plate 13 to reset after the test. A bottom edge of the inner front plate 10, a bottom edge of the inner left side plate 12, and a bottom edge of the inner right side plate 13 are in contact with the bottom plate 5. A test area 18 is defined by the inner rear plate 11, the inner front plate 10, the inner left side plate 12, and the inner right side plate 13. As shown in FIG. 3, pressure sensors 9 are arranged in an array on the bottom plate 5 within the test area 18 to monitor positive pressures received at different positions in the test area 18 and record a pressure distribution after pressure transmission via a loose medium, i.e., to detect patterns of pressure transmission in materials with different structures and thicknesses. A plurality of first hydraulic cylinders 20 and a plurality of first limiting blocks 22 are arranged and spaced apart between the inner front plate 10 and the outer front plate 1, a plurality of second hydraulic cylinders 20 and a plurality of second limiting blocks 22 are arranged and spaced apart between the inner left side plate 12 and the outer left side plate 3, and a plurality of third hydraulic cylinders 20 and a plurality of third limiting blocks 22 are arranged and spaced apart between the inner right side plate 13 and the outer right side plate 4. A support force of each hydraulic cylinder 20 is adjustable, and the hydraulic cylinders 20 can monitor lengths by which the corresponding inner plates are retracted or extended. The corresponding inner and outer plates can be rigidly fixed by the limiting blocks 22 to prevent displacement. Limiting grooves 21 are respectively provided on the inner side surface of the outer front plate 1, the inner side surface of the outer left side plate 3, and the inner side surface of the outer right side plate 4, and on an outer side surface of the inner front plate 10, an outer side surface of the inner left side plate 12, and an outer side surface of the inner right side plate 13. Two ends of each of the plurality of first limiting blocks 22, the plurality of second limiting blocks 22, and the plurality of third limiting blocks 22 are respectively engaged with two respective ones of the limiting grooves 21.

The pressure cover includes a cover body 23, a central disc 24, a pressure column 25, and a pressure plate 26. A shape and a size of the cover body 23 match a shape and a size of the outer side frame. The central disc 24 is arranged at a center of the cover body 23. An upper end and a lower end of the pressure column 25 are respectively connected to a lower end surface of the central disc 24 and an upper end surface of the pressure plate 26. A lifting ring 27 is arranged on an upper top surface of the central disc 24. The pressure plate 26 is located above the test area 18. Four second connecting columns 7 are respectively arranged at middle positions on four edges of a lower end surface of the cover body 23. The four second connecting columns 7 on the lower end surface of the cover body 23 correspond to and are respectively connected to the four first connecting columns 7 of the outer side frame by connecting buckles 8, so that the pressure cover is detachably connected to the outer side frame.

To enable the simulation device to be moved as a whole and to facilitate field testing, a hydraulic pump and a data logger are mounted on the bottom plate 5 between the inner rear plate 11 and the outer rear plate 2.

The simulation device of the present disclosure can separately carry out a consolidation test without lateral deformation, separately carry out a pressure settlement test with a weak constraint in a single direction or without a constraint, separately carry out a pressure settlement test with variable constraints in multiple directions, and separately carry out a stress transfer test.

Preparation Phase of Test: First, the pressure cover is opened through the lifting ring 27. Then, each limiting block 22 is mounted in two respective ones of the limiting grooves 21, and a material is loaded in the test area 18 as designed. Then, the pressure cover is closed and is locked by the connecting buckles 8. Stress transfer under pressure of pressure plates of different shapes, e.g., circular, square, and rectangular pressure plates 26, is simulated by adjusting the shape of the pressure plate 26.

To separately carry out a consolidation test without lateral deformation (i.e., a large-scale conventional consolidation test), a vertical pressure is applied to a material by the pressure column 25. A relationship between the vertical pressure from the pressure column 25, a compression amount of the material, and a duration for which the vertical pressure is applied to the material is recorded.

To separately carry out a pressure settlement test with a weak constraint in a single direction or without a constraint (i.e., simulate a recessed dump), the plurality of first limiting blocks 22 between the inner front plate 10 and the outer front plate 1 are taken out. A pressure upper limit is set for the plurality of first hydraulic cylinders 20 between the inner front plate 10 and the outer front plate 1. A vertical pressure is applied to a material by the pressure column 25. A relationship between the vertical pressure from the pressure column 25, a compression amount of the material, a displacement of the inner front plate 10, pressure values of the pressure sensors 9 on the bottom plate 5, and a duration for which the vertical pressure is applied to the material is recorded.

To separately carry out a pressure settlement test with variable constraints in multiple directions (i.e., simulate a raised dump), the plurality of first limiting blocks 22 between the inner front plate 10 and the outer front plate 1, the plurality of second limiting blocks 22 between the inner left side plate 12 and the outer left side plate 3, and the plurality of third limiting blocks 22 between the inner right side plate 13 and the outer right side plate 4 are taken out. An upper limit is set for support forces from the plurality of first hydraulic cylinders 20 between the inner front plate 10 and the outer front plate 1, the plurality of second hydraulic cylinders 20 between the inner left side plate 12 and the outer left side plate 3, and the plurality of third hydraulic cylinders 20 between the inner right side plate 13 and the outer right side plate 4. A vertical pressure is applied to a material by the pressure column 25. The vertical pressure from the pressure column 25, a compression amount of the material, a pressure received by the inner front plate 10, a horizontal displacement of the inner front plate 10, a pressure received by the inner left side plate 12, a horizontal displacement of the inner left side plate 12, a pressure received by the inner right side plate 13, a horizontal displacement of the inner right side plate 13, displacements of the plurality of first hydraulic cylinders 20, the plurality of second hydraulic cylinders 20 and the plurality of third hydraulic cylinders 20, pressure values of the pressure sensors 9 on the bottom plate 5, and a duration for which the vertical pressure is applied to the material are recorded.

To separately carry out a stress transfer test, the plurality of first limiting blocks 22 between the inner front plate 10 and the outer front plate 1 are taken out or the plurality of second limiting blocks 22 between the inner left side plate 12 and the outer left side plate 3 and the plurality of third limiting blocks 22 between the inner right side plate 13 and the outer right side plate 4 are taken out. A displacement limit is set for the inner front plate 10 or for the inner left side plate 12 and the inner right side plate 13 in advance. A vertical pressure is applied to a material by the pressure column 25. A relationship between the vertical pressure from the pressure column 25, a compression amount of the material, a pressure received by the inner front plate 10 or a pressure received by the inner left side plate 12 and the inner right side plate 13, pressure values of the pressure sensors 9 on the bottom plate 5, and a duration for which the vertical pressure is applied to the material is recorded.

The four tests are independent of each other, and the order in which the above four tests are carried out is not limited. A single simulation device of the present disclosure can carry out a plurality of different tests and simulate a plurality of field environments.

Prediction of dump settlement based on integral method:

When an internal friction angle of the material is greater than a slope angle, the material in an n^{th} unit in a lower part is considered to have no lateral displacement, settlement of the material in the n^{th} unit is calculated using a result of the consolidation test without lateral deformation, with settlement of an (n-1)^{th} differential unit under its own weight being used as a base value for the calculation.

When the internal friction angle of the material is less than the slope angle or bench face angle, the material is considered to be under a weak lateral constraint, the constraint force is a product of a lateral weight of the material and a friction coefficient, and settlement of the material is calculated using a corresponding test result, with settlement of an (n-1)^{th} differential unit under its own weight being used as a base value for the calculation.

For an additional load generated by operation of the simulation device, a lateral constraint of the material is calculated using a method of a pressure settlement test with a weak constraint in a single direction, an overburden pressure of the n^{th} unit is calculated using data of the pressure sensors on the bottom plate that is obtained from a stress transfer test, and then settlement and a horizontal displacement are obtained through a pressure test with a weak lateral constraint.

It will be apparent to those skilled in the art that the present disclosure is not limited to the details of the above-described exemplary embodiments, and the present disclosure may be embodied in other specific forms without departing from the spirit or essential characteristics of the present disclosure. Therefore, the embodiments are to be regarded in all respects as exemplary and non-limiting, and the scope of the present disclosure is to be defined by the appended claims rather than the foregoing description. Hence, all changes falling within the meaning and scope of the equivalents of the claims are intended to be included in the present disclosure. Any reference signs in the claims shall not be construed as limiting the involved claims.

The above descriptions are only preferred embodiments of the present disclosure and are not intended to limit the present disclosure. Any modification, equivalent replacement, or improvement made to the above embodiments based on the technical essence of the present disclosure shall be included in the protection scope of the technical solutions of the present disclosure.

What is claimed is:

1. A simulation device for consolidation and settlement with a variable confinement constraint, comprising an outer front plate, an outer rear plate, an outer left side plate, an outer right side plate, and a bottom plate, wherein a left edge and a right edge of the outer front plate are respectively connected to a front edge of the outer left side plate and a front edge of the outer right side plate, a left edge and a right edge of the outer rear plate are respectively connected to a rear edge of the outer left side plate and a rear edge of the outer right side plate, a bottom edge of the outer front plate, a bottom edge of the outer rear plate, a bottom edge of the outer left side plate, and a bottom edge of the outer right side plate are connected to a top surface of the bottom plate, and the outer front plate, the outer rear plate, the outer left side plate, and the outer right side plate form an outer side frame;
further comprising a pressure cover, an inner front plate, an inner rear plate, an inner left side plate, and an inner right side plate, wherein the inner rear plate is located on an inner side of the outer rear plate, a left edge and a right edge of the inner rear plate are respectively connected to the outer left side plate and the outer right side plate, a bottom edge of the inner rear plate is connected to the top surface of the bottom plate, and the inner rear plate and the outer rear plate are rigidly connected by a supporting column;
inner side-plate slide rails are respectively arranged at positions on an inner side surface of the inner rear plate adjacent to the left edge and the right edge of the inner rear plate and positions on an inner side surface of the outer front plate adjacent to the left edge and the right edge of the outer front plate, and through the inner side-plate slide rails, a front edge and a rear edge of the inner left side plate are respectively connected to the outer front plate and the inner rear plate, and a front edge and a rear edge of the inner right side plate are respectively connected to the outer front plate and the inner rear plate;
inner front-plate slide rails are respectively arranged at a position on an inner side surface of the inner left side plate adjacent to the outer front plate and a position on an inner side surface of the inner right side plate adjacent to the outer front plate, a telescopic cylinder is arranged inside the inner front plate, two telescopic bars are respectively arranged at a left end and a right end of the telescopic cylinder, and the two telescopic bars at the left end and the right end of the telescopic cylinder are respectively connected to the inner left side plate and the inner right side plate through the inner front-plate slide rails;
a bottom edge of the inner front plate, a bottom edge of the inner left side plate, and a bottom edge of the inner right side plate are in contact with the bottom plate, a test area is defined by the inner rear plate, the inner front plate, the inner left side plate, and the inner right side plate, and pressure sensors are arranged on the bottom plate within the test area;
a first hydraulic cylinder and a first limiting block are arranged between the inner front plate and the outer front plate, a second hydraulic cylinder and a second limiting block are arranged between the inner left side plate and the outer left side plate, a third hydraulic cylinder and a third limiting block are arranged between the inner right side plate and the outer right side plate, limiting grooves are respectively provided on the inner side surface of the outer front plate, an inner side surface of the outer left side plate, and an inner side surface of the outer right side plate, and on an outer side surface of the inner front plate, an outer side surface of the inner left side plate, and an outer side surface of the inner right side plate, and two ends of each of the first limiting block, the second limiting block, and the third limiting block are respectively engaged with two respective ones of the limiting grooves; and
the pressure cover comprises a cover body, a central disc, a pressure column, and a pressure plate, a shape and a size of the cover body match a shape and a size of the outer side frame, the central disc is arranged at a center of the cover body, an upper end and a lower end of the pressure column are respectively connected to a lower end surface of the central disc and an upper end surface of the pressure plate, the pressure plate is located above the test area, and the pressure cover is detachably connected to the outer side frame.

2. The simulation device for consolidation and settlement with the variable confinement constraint according to claim 1, wherein a reinforcing column is arranged at each of four inner corners of the outer side frame.

3. The simulation device for consolidation and settlement with the variable confinement constraint according to claim 1, wherein four first connecting columns are respectively arranged at a middle position on the inner side surface of the outer front plate, a middle position on an inner side surface of the outer rear plate, a middle position on the inner side surface of the outer left side plate, and a middle position on the inner side surface of the outer right side plate.

4. The simulation device for consolidation and settlement with the variable confinement constraint according to claim 3, wherein four second connecting columns are respectively arranged at middle positions on four edges of a lower end surface of the cover body, and the four second connecting columns on the lower end surface of the cover body are respectively connected to the four first connecting columns of the outer side frame by connecting buckles.

5. The simulation device for consolidation and settlement with the variable confinement constraint according to claim 1, wherein two telescopic cylinders are provided and spaced apart in a vertical direction, and a plurality of first hydraulic cylinders and a plurality of first limiting blocks are arranged and spaced apart between the inner front plate and the outer front plate, a plurality of second hydraulic cylinders and a plurality of second limiting blocks are arranged and spaced apart between the inner left side plate and the outer left side plate, and a plurality of third hydraulic cylinders and a plurality of third limiting blocks are arranged and spaced apart between the inner right side plate and the outer right side plate.

6. The simulation device for consolidation and settlement with the variable confinement constraint according to claim 1, wherein a lifting ring is arranged on an upper top surface of the central disc.

7. The simulation device for consolidation and settlement with the variable confinement constraint according to claim 1, wherein the pressure sensors are arranged in an array on the bottom plate within the test area.

8. The simulation device for consolidation and settlement with the variable confinement constraint according to claim 1, wherein the pressure plate and the pressure column are connected by a snap- fit, and the pressure plate is circular, square, or rectangular.

9. A simulation method for consolidation and settlement with a variable confinement constraint, comprising:
simulating a consolidation test without lateral deformation: applying a vertical pressure to a material by a pressure column, and recording a relationship between the vertical pressure from the pressure column, a compression amount of the material, and a duration for which the vertical pressure is applied to the material;
simulating a pressure settlement test with a weak constraint in a single direction or without a constraint: taking out a first limiting block between an inner front plate and an outer front plate, setting a pressure upper limit for a first hydraulic cylinder between the inner front plate and the outer front plate, applying a vertical pressure to a material by the pressure column, and recording a relationship between the vertical pressure from the pressure column, a compression amount of the material, a displacement of the inner front plate, pressure values of pressure sensors on a bottom plate, and a duration for which the vertical pressure is applied to the material;
simulating a pressure settlement test with variable constraints in multiple directions: taking out the first limiting block between the inner front plate and the outer front plate, a second limiting block between an inner left side plate and an outer left side plate, and a third limiting block between an inner right side plate and an outer right side plate, setting an upper limit for support forces from the first hydraulic cylinder between the inner front plate and the outer front plate, a second hydraulic cylinder between the inner left side plate and the outer left side plate, and a third hydraulic cylinder between the inner right side plate and the outer right side plate, applying a vertical pressure to a material by the pressure column, and recording the vertical pressure from the pressure column, a compression amount of the material, a pressure received by the inner front plate, a horizontal displacement of the inner front plate, a pressure received by the inner left side plate, a horizontal displacement of the inner left side plate, a pressure received by the inner right side plate, a horizontal displacement of the inner right side plate, displacements of the first hydraulic cylinder, the second hydraulic cylinder and the third hydraulic cylinder, pressure values of the pressure sensors on the bottom plate, and a duration for which the vertical pressure is applied to the material; and
simulating a stress transfer test: taking out the first limiting block between the inner front plate and the outer front plate or taking out the second limiting block between the inner left side plate and the outer left side plate and the third limiting block between the inner right side plate and the outer right side plate, setting a displacement limit for the inner front plate or for the inner left side plate and the inner right side plate in advance, applying a vertical pressure to a material by the pressure column, recording a relationship between the vertical pressure from the pressure column, a compression amount of the material, a pressure received by the inner front plate or a pressure received by the inner left side plate and the inner right side plate, pressure values of the pressure sensors on the bottom plate, and a duration for which the vertical pressure is applied to the material.
